(19) United States
(12) Patent Application Publication
(10) Pub. No.: US 2013/0096429 A1
(43) Pub. Date: Apr. 18, 2013

(54) ULTRASONIC DIAGNOSIS APPARATUS, OPERATION METHOD OF THE SAME, AND COMPUTER READABLE RECORDING MEDIUM

(71) Applicant: Hiromasa NOGUCHI, Tokyo (JP)

(72) Inventor: Hiromasa NOGUCHI, Tokyo (JP)

(73) Assignee: OLYMPUS MEDICAL SYSTEMS CORP., Tokyo (JP)

(21) Appl. No.: 13/562,797

(22) Filed: Jul. 31, 2012

(30) Foreign Application Priority Data
Nov. 11, 2010 (JP) 2010-253287

(51) Int. Cl. A61B 8/08; A61B 8/14; A61B 8/00

(52) U.S. Cl. CPC A61B 8/5223; A61B 8/08; A61B 8/5207; A61B 8/463; A61B 8/14
USPC 600/438

(57) ABSTRACT

An ultrasonic diagnosis apparatus includes: a frequency analyzing unit that calculates a frequency spectrum of a received ultrasonic wave; a frequency band setting unit that sets at least an upper limit frequency for use in approximating the calculated frequency spectrum to a predetermined frequency according to a receiving depth of an ultrasonic wave; a feature data extracting unit that extracts feature data of the frequency spectrum by approximating a frequency spectrum of the frequency band; a storage unit that stores feature data of a frequency spectrum extracted based on ultrasonic waves reflected off known specimens in association with tissue characteristics of the known specimens; and a tissue characteristic determining unit that determines a tissue characteristic in a predetermined area in the specimen using feature data stored in the storage unit in association with tissue characteristics of the known specimens and the feature data extracted at the feature data extracting unit.

### CROSS REFERENCES TO RELATED APPLICATIONS

This application is a continuation of PCT international application Ser. No. PCT/JP2011/076028 filed on Nov. 11, 2011 which designates the United States, incorporated herein by reference, and which claims the benefit of priority from Japanese Patent Applications No. 2010-253287, filed on Nov. 11, 2010, incorporated herein by reference.

### BACKGROUND OF THE INVENTION

1. Field of the Invention

The present invention relates to an ultrasonic diagnosis apparatus, an operation method of an ultrasonic diagnosis apparatus, and an operation program of an ultrasonic diagnosis apparatus that determine the tissue characteristic of a specimen using ultrasonic waves.

2. Description of the Related Art

Heretofore, a technique called ultrasonic elastography is known as an examination technique for breast cancer or the like using ultrasonic waves (see International Publication No. WO/2005/122906, for example). The ultrasonic elastography is a technique using the fact that the hardness of cancer or tumor tissue in a living body is varied depending on the advancement of illness or living bodies. In this technique, in a state in which an examination location is externally pressed, ultrasonic waves are used to measure the strain value or the modulus of elasticity of human tissue at the examination location, and this measured result is displayed as a tomogram.

### SUMMARY OF THE INVENTION

An ultrasonic diagnosis apparatus according to the present invention transmits an ultrasonic wave to a specimen and receives an ultrasonic wave reflected off the specimen for determining a tissue characteristic of the specimen based on a received ultrasonic wave, the ultrasonic diagnosis apparatus including: a frequency analyzing unit configured to calculate a frequency spectrum by analyzing a frequency of a received ultrasonic wave; a frequency band setting unit configured to set at least an upper limit frequency of a frequency band for use in approximating the frequency spectrum calculated at the frequency analyzing unit to a predetermined frequency according to a receiving depth of an ultrasonic wave; a feature data extracting unit configured to extract feature data of the frequency spectrum by approximating a frequency spectrum of the frequency band set at the frequency band setting unit; a storage unit configured to store feature data of a frequency spectrum extracted based on ultrasonic waves reflected off a plurality of known specimens in association with tissue characteristics of the plurality of known specimens; and a tissue characteristic determining unit configured to determine a tissue characteristic in a predetermined area in the specimen using feature data stored in the storage unit in association with tissue characteristics of the plurality of known specimens and the feature data extracted at the feature data extracting unit.

An operation method of an ultrasonic diagnosis apparatus according to the present invention transmits an ultrasonic wave to a specimen and receives an ultrasonic wave reflected off the specimen for determining a tissue characteristic of the specimen based on a received ultrasonic wave, the operation method including: calculating a frequency spectrum at a frequency analyzing unit by analyzing a frequency of a received ultrasonic wave; setting at least an upper limit frequency of a frequency band for use in approximating the calculated frequency spectrum to a predetermined frequency according to a receiving depth of an ultrasonic wave; extracting feature data of the frequency spectrum at a feature data extracting unit by approximating a frequency spectrum of the set frequency band; and determining a tissue characteristic in a predetermined area in the specimen at a tissue characteristic determining unit using feature data read out of a storage unit storing feature data of a frequency spectrum extracted based on ultrasonic waves reflected off a plurality of known specimens in association with tissue characteristics of the plurality of known specimens and the feature data extracted at the feature data extracting unit.

A non-transitory computer readable recording medium according to the present invention has an executable program recorded thereon, wherein the program instructs a processor to perform: calculating a frequency spectrum at a frequency analyzing unit by analyzing a frequency of a received ultrasonic wave; setting at least an upper limit frequency of a frequency band for use in approximating the calculated frequency spectrum to a predetermined frequency according to a receiving depth of an ultrasonic wave; extracting feature data of the frequency spectrum at a feature data extracting unit by approximating a frequency spectrum of the set frequency band; and determining a tissue characteristic in a predetermined area in the specimen at a tissue characteristic determining unit using feature data read out of a storage unit storing feature data of a frequency spectrum extracted based on ultrasonic waves reflected off a plurality of known specimens in association with tissue characteristics of the plurality of known specimens and the feature data extracted at the feature data extracting unit.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram of the configuration of an ultrasonic diagnosis apparatus according to a first embodiment of the present invention;

FIG. 2 is a schematic diagram of an exemplary frequency band table stored in the ultrasonic diagnosis apparatus according to the first embodiment of the present invention;

FIG. 3 is a flowchart of the outline of a process performed by the ultrasonic diagnosis apparatus according to the first embodiment of the present invention;

FIG. 4 is a diagram of an exemplary display of a B mode image on a display unit of the ultrasonic diagnosis apparatus according to the first embodiment of the present invention;

FIG. 5 is a flowchart of the outline of a process performed by a frequency analyzing unit of the ultrasonic diagnosis apparatus according to the first embodiment of the present invention;

FIG. 6 is a schematic diagram of a data array of a single sound ray;

FIG. 7 is a diagram of an exemplary frequency spectrum (a first example) calculated at the frequency analyzing unit of the ultrasonic diagnosis apparatus according to the first embodiment of the present invention;

FIG. 8 is a diagram of an exemplary frequency spectrum (a second example) calculated at the frequency analyzing unit of the ultrasonic diagnosis apparatus according to the first embodiment of the present invention;

FIG. 9 is a diagram of an exemplary frequency spectrum (a third example) calculated at the frequency analyzing unit of the ultrasonic diagnosis apparatus according to the first embodiment of the present invention;

FIG. 10 is a flowchart of the outline of a process performed by a tissue characteristic determining unit of the ultrasonic diagnosis apparatus according to the first embodiment of the present invention;

FIG. 11 is a diagram of an exemplary feature data space set by the tissue characteristic determining unit of the ultrasonic diagnosis apparatus according to the first embodiment of the present invention;

FIG. 12 is a diagram of an exemplary display of a determined result display image displayed on the display unit of the ultrasonic diagnosis apparatus according to the first embodiment of the present invention;

FIG. 13 is a diagram of another exemplary display of a determined result display image displayed on the display unit of the ultrasonic diagnosis apparatus according to the first embodiment of the present invention; and

FIG. 14 is a diagram of the outline of a process for determining a tissue characteristic performed at a tissue characteristic determining unit of an ultrasonic diagnosis apparatus according to a fourth embodiment of the present invention.

### DETAILED DESCRIPTION OF THE PREFERRED EMBODIMENTS

In the following, modes for carrying out the present invention (in the following, referred to as “embodiments”) will be described with reference to the accompanying drawings.

### First Embodiment

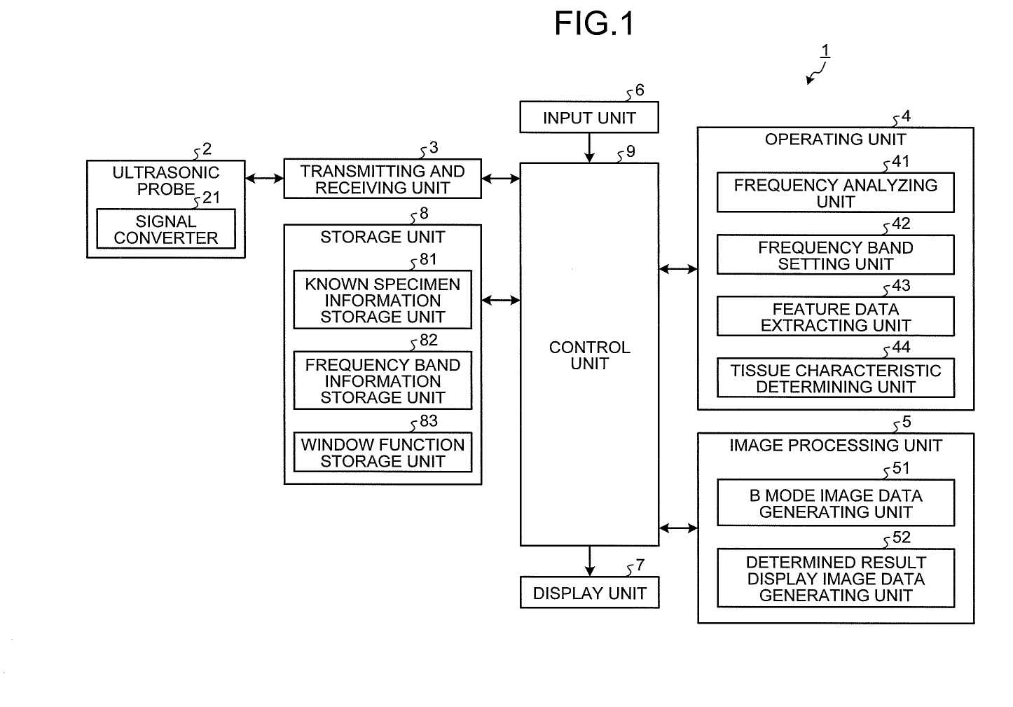

FIG. 1 is a block diagram of the configuration of an ultrasonic diagnosis apparatus according to a first embodiment of the present invention. An ultrasonic diagnosis apparatus 1 illustrated in FIG. 1 is an apparatus that determines the tissue characteristic of a specimen, which is a diagnosis subject, using ultrasonic waves.

The ultrasonic diagnosis apparatus 1 includes an ultrasonic probe 2 that externally outputs ultrasonic pulses and receives externally reflected ultrasonic echoes, a transmitting and receiving unit 3 that transmits and receives electric signals with the ultrasonic probe 2, an operating unit 4 that applies a predetermined arithmetic operation to electrical echo signals converted from ultrasonic echoes, an image processing unit 5 that generates image data corresponding to the electrical echo signals converted from the ultrasonic echoes, an input unit 6 implemented using an interface such as a keyboard, a mouse, and a touch panel to accept inputs of various items of information, a display unit 7 implemented using a display panel formed of liquid crystals or an organic electroluminescence to display various items of information including images created at the image processing unit 5, a storage unit 8 that stores various items of information including information about the tissue characteristics of known specimens, and a control unit 9 that controls the operation of the ultrasonic diagnosis apparatus 1.

The ultrasonic probe 2 includes a signal converter 21 that converts electrical pulse signals received from the transmitting and receiving unit 3 into ultrasonic pulses (sound pulse signals) and converts ultrasonic echoes reflected off an external specimen into electrical echo signals. The ultrasonic probe 2 may be a device that mechanically scans an ultrasonic oscillator, or may be a device that electronically scans a plurality of ultrasonic oscillators.

The transmitting and receiving unit 3 is electrically connected to the ultrasonic probe 2 to send pulse signals to the ultrasonic probe 2 and to receive echo signals from the ultrasonic probe 2. More specifically, the transmitting and receiving unit 3 generates pulse signals based on preset waveforms and transmitting timing, and sends the generated pulse signals to the ultrasonic probe 2. Moreover, the transmitting and receiving unit 3 applies processing such as amplifying and filtering to the received echo signals, and then subjects the echo signals to A/D conversion to generate and output digital RF signals. Incidentally, in the case where the ultrasonic probe 2 is a device that electronically scans a plurality of ultrasonic oscillators, the transmitting and receiving unit 3 includes a multichannel circuit for combining beams corresponding to the plurality of ultrasonic oscillators.

The operating unit 4 includes a frequency analyzing unit 41 that applies fast Fourier transform (FFT) to the digital RF signals outputted from the transmitting and receiving unit 3 for analyzing the frequency of the echo signals, a frequency band setting unit 42 that sets a frequency band for use in approximating a frequency spectrum (a power spectrum) calculated at the frequency analyzing unit 41, a feature data extracting unit 43 that approximates the frequency spectrum of the frequency band set by the frequency band setting unit 42 for extracting the feature data of the frequency spectrum, and a tissue characteristic determining unit 44 that determines a tissue characteristic in a predetermined area in a specimen using the feature data extracted at the feature data extracting unit 43.

The frequency analyzing unit 41 applies fast Fourier transform to an FFT data group formed of a predetermined data volume with respect to sound rays (line data) for calculating a frequency spectrum. The frequency spectrum shows different tendencies depending on the tissue characteristic of a specimen. This is because the frequency spectrum has the correlation with the size, density, acoustic impedance, or the like of a specimen as a scatterer that scatters ultrasonic waves.

The frequency band setting unit 42 reads and makes reference to a frequency band table (described later) stored in the storage unit 8 out of the storage unit 8 for setting a frequency band. The reason why the setting of the frequency band is changed for every receiving depth is that in the case of ultrasonic waves, a higher radio frequency component is attenuated faster and it is likely that the valid information of the radio frequency component is lost from echo signals received from a location where the receiving depth is deep and invalid information remains. In view of this point, in the first embodiment, the frequency band is set in such a way that the bandwidth is made narrower and the maximum frequency is made smaller as the receiving depth is deeper.

The feature data extracting unit 43 approximates the frequency spectrum in a linear expression by regression analysis, and extracts feature data characterizing the approximated linear expression. More specifically, the feature data extracting unit 43 calculates a slope a and an intercept b of the linear expression by regression analysis, and calculates the intensity of a characteristic frequency in the frequency band of the frequency spectrum. In the first embodiment, the feature data extracting unit 43 calculates intensity (Mid-band fit) c=af_{MID}+b in a center frequency f_{MID}=(f_{Low}+f_{HIGH})/2. However, this is merely an example. “The intensity” here means any one of parameters such as voltage, electric power, sound pressure, and acoustic energy.

Among three items of feature data, the slope a has the correlation with the size of a scatterer for ultrasonic waves, and it is generally considered that a larger scatterer has a smaller slope value. Moreover, the intercept b has the correlation with the size of a scatterer, a difference in acoustic impedance, the density (concentration) of a scatterer, or the like. More specifically, it is considered that the intercept b has a larger value as a scatterer is larger, has a larger value as acoustic impedance is larger, and has a larger value as the density (the concentration) of a scatterer is larger. An intensity c in the center frequency f_{MID} (in the following, simply referred to as “intensity”) is an indirect parameter derived from the slope a and the intercept b, and gives a spectrum intensity at the center in an effective frequency band. Therefore, it is considered that the intensity c has the correlation with the brightness of a B mode image to a certain degree, in addition to the correlation with the size of a scatterer, a difference in acoustic impedance, and the density of a scatterer. Incidentally, an approximate polynomial equation calculated at the feature data extracting unit 43 is not limited to a linear expression, and a second-order or higher-order approximate polynomial equation may be used.

The tissue characteristic determining unit 44 calculates the average and standard deviation of the feature data of the frequency spectrum extracted at the feature data extracting unit 43 for every item of feature data. The tissue characteristic determining unit 44 determines a tissue characteristic in a predetermined area in a specimen using a difference between the calculated average and standard deviation and the average and standard deviation of the feature data of the frequency spectra of known specimens stored in the storage unit 8. “A predetermined area” here is referred to as an area in an image specified through the input unit 6 by the operator of the ultrasonic diagnosis apparatus 1 who sees the image created at the image processing unit 5 (in the following, referred to as “the area of interest”). Moreover, “a tissue characteristic” here is any one of cancer, endocrinoma, mucinous tumor, normal tissue, and vascular, for example. Incidentally, in the case where a specimen is a pancreas, tissue characteristics also include chronic pancreatitis, autoimmune pancreatitis, etc.

The average and standard deviation of feature data calculated at the tissue characteristic determining unit 44 reflect changes at cell level such as nucleus swell and heteromorphic nucleus and organic changes such as fiber hyperplasia in interstitial tissue and the replacement of parenchyma with fiber, showing unique values depending on tissue characteristics. Therefore, the average and standard deviation of feature data as described above are used to accurately determine a tissue characteristic in a predetermined area in a specimen.

The image processing unit 5 includes a B mode image data generating unit 51 that generates B mode image data showing brightness converted from the amplitudes of echo signals, and a determined result display image data generating unit 52 that uses data outputted from the B mode image data generating unit 51 and data outputted from the operating unit 4 to generate determined result display image data showing the determined result of a tissue characteristic in the area of interest and information about the determined result.

The B mode image data generating unit 51 applies signal processing to digital signals using publicly known techniques such as bandpass filtering, logarithm transformation, gain processing, and contrast processing, and generates B mode image data by data reduction according to data step width determined depending on the display range of an image at the display unit 7, for example.

The determined result display image data generating unit 52 generates determined result display image data including the determined result of the tissue characteristic in the area of interest and a tissue characteristic emphasized image that emphasizes the tissue characteristic using the B mode image data generated at the B mode image data generating unit 51, the feature data calculated at the feature data extracting unit 43, and the determined result determined at the tissue characteristic determining unit 44.

The storage unit 8 includes a known specimen information storage unit 81 that stores information about a known specimen, a frequency band information storage unit 82 that stores frequency band information defined according to the receiving depth of ultrasonic waves, and a window function storage unit 83 that stores window functions for use in frequency analysis performed at the frequency analyzing unit 41.

The known specimen information storage unit 81 stores the feature data of the frequency spectrum extracted with respect to a known specimen in association with the tissue characteristic of the known specimen. Moreover, the known specimen information storage unit 81 stores the averages and standard deviations calculated for every group sorted based on the tissue characteristics of the known specimens with respect to the feature data of the frequency spectra related to known specimens together with the entire feature data of the known specimens. Here, the feature data of the known specimens is extracted by processing similar to processing in the first embodiment. However, it is unnecessary to extract the feature data of the known specimens in the ultrasonic diagnosis apparatus 1. Desirably, information about known specimens stored in the known specimen information storage unit 81 is highly reliable information about tissue characteristics.

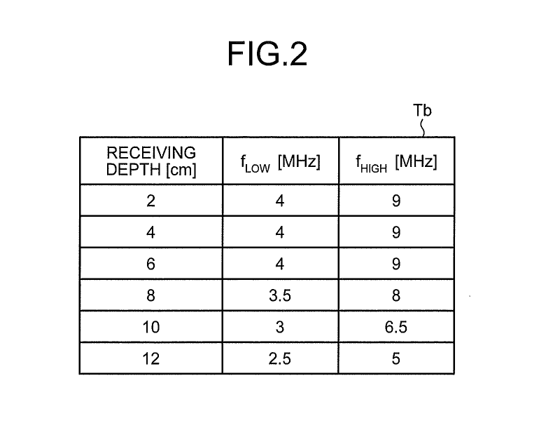

FIG. 2 is a schematic diagram of a frequency band table as frequency band information stored in the frequency band information storage unit 82. A frequency band table Tb illustrated in FIG. 2 illustrates the minimum frequency (flow) and the maximum frequency (f_{HIGH}) for the individual receiving depths of ultrasonic waves. In the frequency band table Tb, the bandwidth f_{HIGH}-f_{LOW} is narrow and the maximum frequency f_{HIGH} is smaller, as the receiving depth is deeper. Moreover, in the case where the receiving depth is relatively shallow (2 to 6 cm in FIG. 2), the frequency band is not changed in the frequency band table Tb because the influence of attenuation is small. In contrast to this, in the case where the receiving depth is relatively deep (8 to 12 cm in FIG. 2), the band is made narrower and moved to the low frequency side because the influence of attenuation becomes large. This frequency band table Tb is used to extract only signals having valid information for imaging.

The window function storage unit 83 stores at least one of window functions such as Hamming, Hanning, and Blackman, or stores a plurality of window functions. The storage unit 8 is implemented using ROM on which the operation program of the ultrasonic diagnosis apparatus according to the first embodiment, a program to start a predetermined OS, or the like is stored in advance and RAM on which arithmetic operation parameters, data for processes, or the like are stored, for example.

The components other than the ultrasonic probe 2 of the ultrasonic diagnosis apparatus 1 having functionalities and configurations described above are implemented using a computer including a CPU having an arithmetic operation functionality and a control functionality. The CPU included in the ultrasonic diagnosis apparatus 1 executes arithmetic operation processing related to an operation method of the ultrasonic diagnosis apparatus according to the first embodiment by reading information memorized and stored in the storage unit 8 and various programs including the operation program of the ultrasonic diagnosis apparatus described above out of the storage unit 8.

Incidentally, the operation program of the ultrasonic diagnosis apparatus according to the first embodiment may be stored on a computer readable recording medium such as a hard disk, flash memory, a CD-ROM, a DVD-ROM, and a flexible disk, for wide distribution.

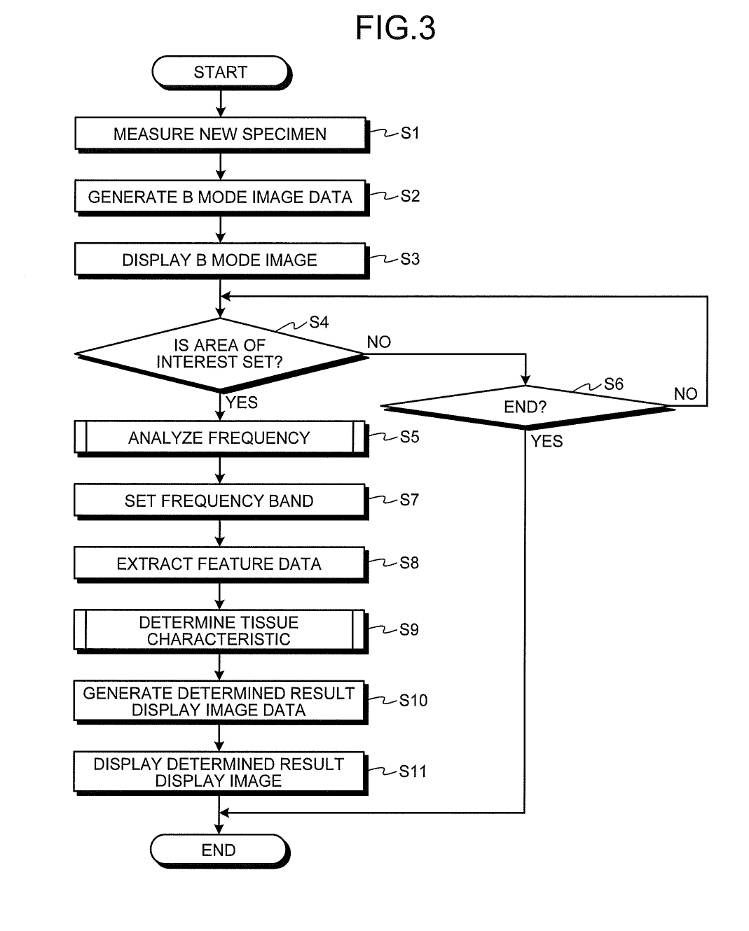

FIG. 3 is a flowchart of the outline of a process performed by the ultrasonic diagnosis apparatus 1 having the configuration described above. In FIG. 3, in the ultrasonic diagnosis apparatus 1, first, the ultrasonic probe 2 is used to measure a new specimen (Step S1). After the measurement, the B mode image data generating unit 51 generates B mode image data (Step S2).

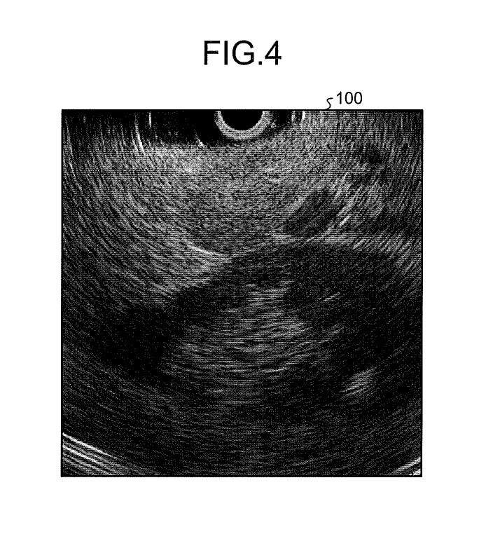

Subsequently, the control unit 9 performs control to display a B mode image corresponding to the B mode image data generated at the B mode image data generating unit 51 on the display unit 7 (Step S3). FIG. 4 is a diagram of an exemplary display of a B mode image on the display unit 7. A B mode image 100 illustrated in FIG. 4 is a gray scale image in which R (red), G (Green), and B (blue) values, which are variables in the case of adopting an RGB color system for a color space, are matched.

After the display, in the case where the area of interest is set through the input unit 6 (Step S4: Yes), the frequency analyzing unit 41 analyzes frequencies by the FFT operation to calculate a frequency spectrum (Step S5). In Step S5, the entire area of the image may be set as the area of interest. On the other hand, in the case where the area of interest is not set (Step S4: No), the ultrasonic diagnosis apparatus 1 ends processing when an instruction to end processing is inputted through the input unit 6 (Step S6: Yes). On the contrary, in the case where the area of interest is not set (Step S4: No), the ultrasonic diagnosis apparatus 1 returns to Step S4 when an instruction to end processing is not inputted through the input unit 6 (Step S6: No).

Here, the process performed at the frequency analyzing unit 41 (Step S5) will be described in detail with reference to a flowchart in FIG. 5. First, the frequency analyzing unit 41 sets a sound ray number L of a sound ray for a subject of analysis to an initial value L₀ (Step S21). The initial value L₀ may be allocated to a sound ray that the transmitting and receiving unit 3 first receives, for example, or may be allocated to a sound ray corresponding to one of sound rays at right and left boundary locations of the area of interest set through the input unit 6.

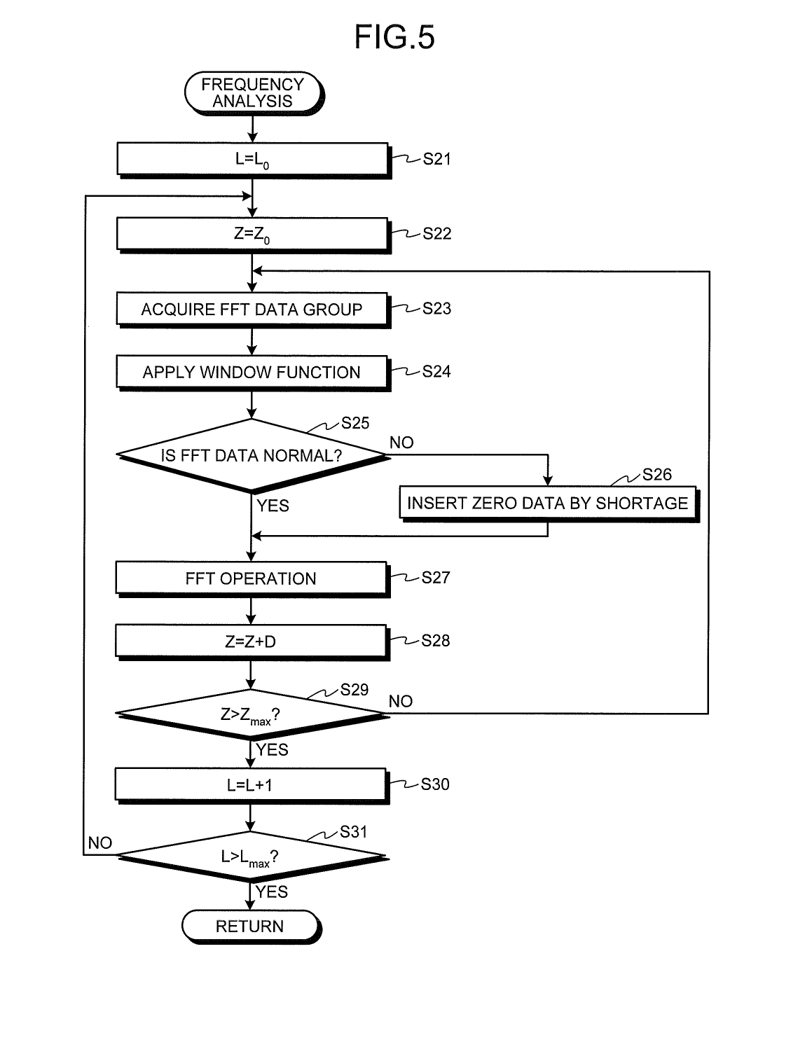

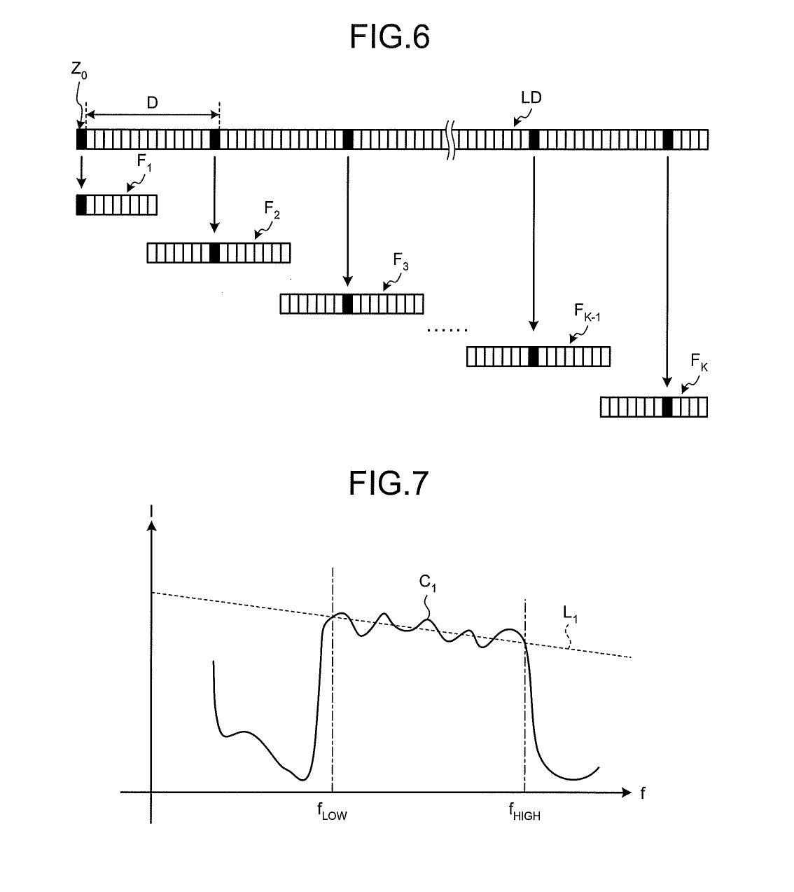

Subsequently, the frequency analyzing unit 41 calculates all frequency spectra at a plurality of data locations set on a single sound ray. First, the frequency analyzing unit 41 sets an initial value Z₀ at a data location Z that represents a sequence of a data group (an FFT data group) acquired for the FFT operation (Step S22). FIG. 6 is a schematic diagram of the data array of a single sound ray. In a sound ray LD illustrated in FIG. 6, a white or black rectangle means an item of data. The sound ray LD is discrete sound rays at time intervals corresponding to a sampling frequency in A/D conversion performed at the transmitting and receiving unit 3 (50 MHz, for example). FIG. 6 illustrates the case where the first item of data of the sound ray LD is set to the initial value Z₀ at the data location Z. Incidentally, FIG. 6 is merely one example, and the location of the initial value Z₀ can be set freely. For example, the data location Z corresponding to the top end location in the area of interest may be set to the initial value Z₀.

After the setting, the frequency analyzing unit 41 acquires an FFT data group at the data location Z (Step S23), and applies a window function stored in the window function storage unit 83 to the acquired FFT data group (Step S24). The window function is thus applied to the FFT data group to avoid a discontinuous FFT data group at the boundary and to prevent artifacts from occurring.

Subsequently, the frequency analyzing unit 41 determines whether the FFT data group at the data location Z is a normal data group (Step S25). Here, the FFT data group is necessary to have a data number of a power of two. In the following, the data number of the FFT data group is 2ⁿ (n is a positive integer). A normal FFT data group means that the data location Z is located at the 2ⁿ⁻¹th location from the front in the FFT data group. In other words, a normal FFT data group means that there are 2ⁿ⁻¹−1 (=N) items of data on the front side of the data location Z and there are 2ⁿ⁻¹ (=M) items of data on the rear side of the data location Z. In the case illustrated in FIG. 6, FFT data groups F₂, F₃, and F_{K-1} are normal, but FFT data groups F₁ and F_{K} are faulty, where n=4 (N=7, M=8) in FIG. 6.

As the result of determination in Step S25, in the case where the FFT data group at the data location Z is normal (Step S25: Yes), the frequency analyzing unit 41 moves to Step S27 described later.

As the result of determination in Step S25, in the case where the FFT data group at the data location Z is faulty (Step S25: No), the frequency analyzing unit 41 inserts zero data by the amount of shortage to generate a normal FFT data group (Step S26). A window function is applied to the FFT data group determined as faulty in Step S25, before adding zero data. Therefore, discontinuous data does not occur even though zero data is inserted into the FFT data group. After Step S26, the frequency analyzing unit 41 moves to Step S27 described below.

In Step S27, the frequency analyzing unit 41 performs the FFT operation using the FFT data group to acquire a frequency spectrum (Step S27).

Subsequently, the frequency analyzing unit 41 adds a predetermined data step width D at the data location Z, and calculates a data location Z of an FFT data group for the subsequent analysis (Step S28). Desirably, the data step width D here is matched with the data step width for use in generating B mode image data at the B mode image data generating unit 51. However, in the case where it is desired to reduce the arithmetic operation amount in the frequency analyzing unit 41, a value greater than the value of the data step width used at the B mode image data generating unit 51 may be set. FIG. 6 illustrates the case of D=15.

After the calculation, the frequency analyzing unit 41 determines whether the data location Z is greater than a last data location Zₘₐₓ (Step S29). Here, the last data location Zₘₐₓ may be the data length of the sound ray LD or may be a data location corresponding to the lower end of the area of interest. As the result of determination, in the case where the data location Z is greater than the last data location Zₘₐₓ (Step S29: Yes), the frequency analyzing unit 41 increments the sound ray number L by one (Step S30). On the other hand, in the case where the data location Z is the last data location Zₘₐₓ or less (Step S29: No), the frequency analyzing unit 41 returns to Step S23. As decried above, the frequency analyzing unit 41 applies the FFT operation to [{(Zₘₐₓ−Z₀)/D}+1] (=K) of FFT data groups for a single sound ray LD. Here, [X] is a maximum integer not greater than X.

In the case where the sound ray number L after incremented in Step S30 is greater than the last sound ray number Lₘₐₓ (Step S31: Yes), the frequency analyzing unit 41 returns to the main routine illustrated in FIG. 2. On the other hand, in the case where the sound ray number L after incremented in Step S30 is the last sound ray number Lₘₐₓ or less (Step S31: No), the frequency analyzing unit 41 returns to Step S22.

As decried above, the frequency analyzing unit 41 performs the FFT operation at K times for (Lₘₐₓ−L₀+1) of sound rays. Incidentally, the last sound ray number Lₘₐₓ may be allocated to the last sound ray received at the transmitting and receiving unit 3, for example, or may be allocated to a sound ray corresponding to any one of sound rays at right and left boundary locations of the area of interest. In the following, suppose that a total number (Lₘₐₓ−L0+1) of the FFT operation×K is P; the FFT operation is applied to all the sound rays at the frequency analyzing unit 41.

Subsequent to the frequency analysis process in Step S5 described above, the frequency band setting unit 42 sets a frequency band to the individual receiving depths of ultrasonic waves with reference to the frequency band table Tb stored in the frequency band information storage unit 82 (Step S7). Incidentally, the process performed at the frequency band setting unit 42 may be performed in parallel with the process performed at the frequency analyzing unit 41, or may be performed prior to the process performed at the frequency analyzing unit 41.

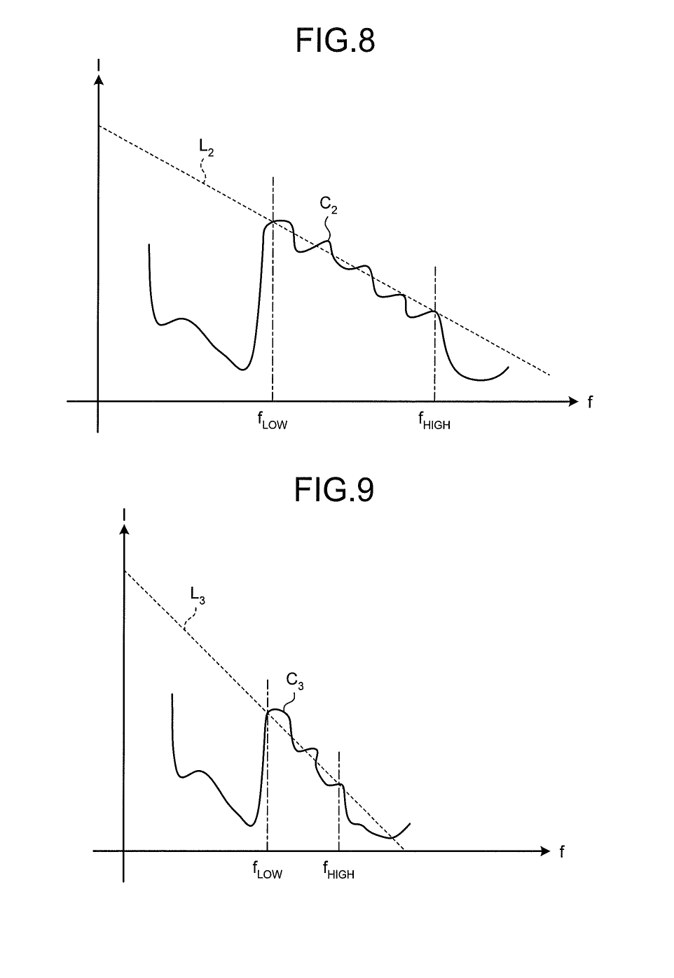

FIGS. 7 to 9 are diagrams of the frequency spectra calculated at the frequency analyzing unit 41 and frequency bands set by the frequency band setting unit 42 with respect to the ultrasonic waves in different receiving depths. In FIGS. 7 to 9, a horizontal axis f expresses the frequency, and a vertical axis I expresses the intensity. More specifically, FIG. 7 is the case where the receiving depth is 2 cm, FIG. 8 is the case where the receiving depth is 8 cm, and FIG. 9 is the case where the receiving depth is 12 cm. In frequency spectrum curves C₁, C₂ and C₃ illustrated in FIGS. 7, 8, and 9, respectively, a lower limit frequency f_{LOW} and an upper limit frequency f_{HIGH} of receiving the frequency spectrum have values set for every depth at the frequency band setting unit 42 based on the frequency band table Tb. In FIG. 7, f_{LOW}=4 (MHz), and f_{HIGH}=9 (MHz). Moreover, in FIG. 8, f_{LOW}=3.5 (MHz), and f_{HIGH}=8 (MHz). Furthermore, in FIG. 9, f_{LOW}=2.5 (MHz), and f_{HIGH}=5 (MHz). It is noted that straight lines L₁, L₂, and L₃ illustrated in FIGS. 7, 8, and 9, respectively, will be described in a feature data extraction process, described later. In the first embodiment, the curves and the straight lines are formed of sets of discrete points. The curves and the straight lines are similarly formed of sets of discrete points also in embodiments described later.

After Step S7, the feature data extracting unit 43 applies regression analysis to P frequency spectra calculated at the frequency analyzing unit 41 in the frequency band set by the frequency band setting unit 42 for extracting feature data (Step S8). More specifically, the feature data extracting unit 43 calculates the slope a, the intercept b, and the intensity c, which are three items of feature data, by calculating a linear expression to approximate the frequency spectrum of the frequency band f_{LOW}<f<f_{HIGH} by regression analysis. The straight lines L₁, L₂, and L₃ illustrated in FIGS. 7, 8, and 9, respectively, are regression lines obtained by performing the feature data extraction process on the frequency spectrum Curves C₁, C₂ and C₃ in Step S8.

After the calculation, the tissue characteristic determining unit 44 determines a tissue characteristic in the area of interest of the specimen based on the feature data extracted at the feature data extracting unit 43 and known specimen information stored in the known specimen information storage unit 81 (Step S9).

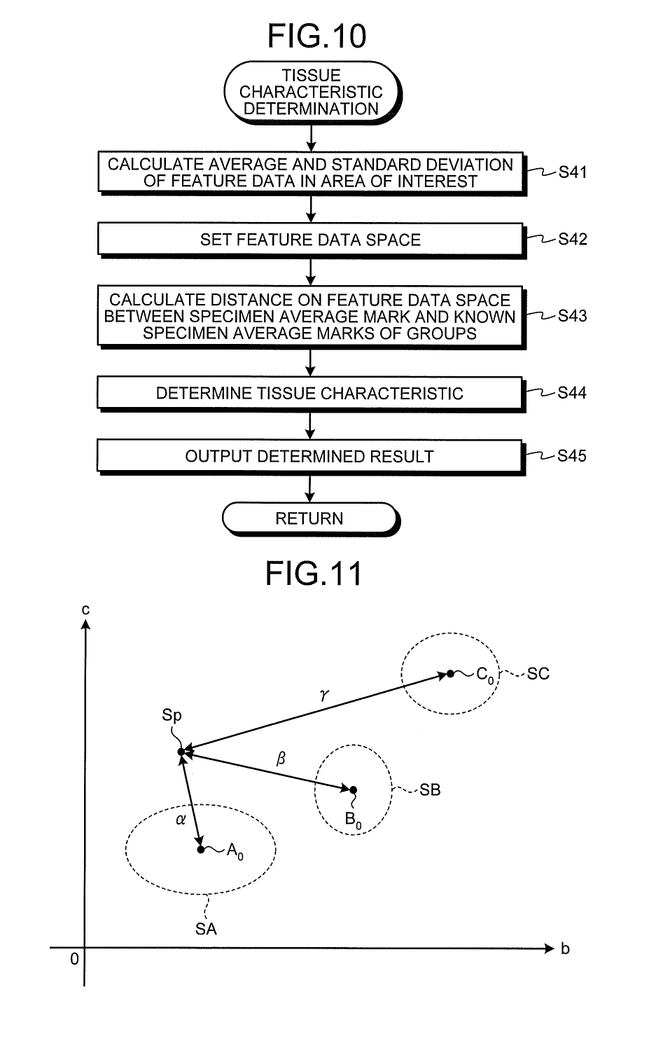

Here, the process performed at the tissue characteristic determining unit 44 (Step S9) will be described in detail with reference to a flowchart in FIG. 10. First, the tissue characteristic determining unit 44 calculates the averages and standard deviations of the slope a, the intercept b, and the intensity c of Q (≦P) sets of FFT data groups located in the area of interest (Step S41).

Subsequently, the tissue characteristic determining unit 44 sets a feature data space for use in determining a tissue characteristic (Step S42). In the first embodiment, there are two independent parameters among the slope a, the intercept b, and the intensity c, which are three items of feature data. Therefore, a two-dimensional space having given two items of feature data as components among three items of feature data can be set to a feature data space. Moreover, a linear space having a single item of feature data as a component among three items of feature data can also be set to a feature data space. In Step S42, it is considered that the feature data space to be set is predetermined. However, the operator may select a desired feature data space through the input unit 6.

FIG. 11 is a diagram of an exemplary feature data space set by the tissue characteristic determining unit 44. In a feature data space illustrated in FIG. 11, the horizontal axis expresses the intercept b, and the vertical axis expresses the intensity c. A point Sp illustrated in FIG. 11 illustrates a point having the averages of the intercept b and the intensity c of the frequency spectra of the FFT data groups included in the area of interest of the specimen calculated at the feature data extracting unit 43 in Step S41 as the coordinates of the feature data space (in the following, this point is referred to as “a specimen average mark”). Moreover, areas SA, SB, and SC illustrated in FIG. 11 are groups that the tissue characteristics of known specimens stored in the known specimen information storage unit 81 are A, B, and C, respectively. In the case illustrated in FIG. 11, three groups SA, SB, and SC exist in areas not overlapping with the other groups on the feature data space.

In the first embodiment, also in determining the feature data of a known specimen, tissue characteristics are sorted and determined using the feature data of a frequency spectrum obtained by approximating the frequency spectrum in a frequency band determined according to the receiving depth of ultrasonic waves as an index, so that tissue characteristics different from each other can be distinguished. Particularly, in the first embodiment, since the frequency band is determined in such a way that the bandwidth is narrower and the maximum frequency is smaller as the receiving depth is deeper, the involvement of a radio frequency component with a large attenuation can be eliminated as compared with the case where the frequency band is made constant to extract feature data regardless of the receiving depth. As a result, the areas of groups in the feature data space can be obtained in a state in which the areas are more clearly separated form each other.

After Step S42, the tissue characteristic determining unit 44 calculates distances α, β, and γ on the feature data space between the specimen average mark Sp and points A₀, B₀, and C₀ having the averages of the intercept b and the intensity c of the frequency spectra of the FFT data groups included in the groups SA, SB, and SC as the coordinates of the feature data space (in the following, these points are referred to as “known specimen average marks”) (Step S43). Here, in the case where the scales of a b-axis component and a c-axis component are greatly different from each other in the feature data space, desirably, weighting is appropriately performed for making the involvement of the distances almost equal.

Subsequently, the tissue characteristic determining unit 44 determines a tissue characteristic at the specimen average mark Sp based on the distances calculated in Step S43 (Step S44). In the case of FIG. 11, the distance a is the shortest. Therefore, the tissue characteristic determining unit 44 determines that the tissue characteristic of the specimen is A. It is noted that in the case where the specimen average mark Sp is extremely separated from the known specimen average marks A₀, B₀, and C₀, the confidence level of the determined result of the tissue characteristic is low even though the minimum values of the distances α, β, and γ are determined. Thus, in the case where the distances α, β, and γ are greater than a predetermined threshold, the tissue characteristic determining unit 44 may output an error signal. Moreover, in the case where two or more of the distances α, β, and γ take a minimum value, the tissue characteristic determining unit 44 may select all the tissue characteristics corresponding to the minimum values as candidates, or may select any one of tissue characteristics according to a predetermined criterion. In the case of the latter, such a method can be named that the priority level of highly malignant tissue characteristics such as cancer is set higher, for example. Moreover, in the case where two or more of the distances α, β, and γ take minimum values, the tissue characteristic determining unit 44 may output an error signal.

After the determination, the tissue characteristic determining unit 44 outputs the result of the distances calculated in Step S43 and the determined result in Step S44 (Step S45). The tissue characteristic determination process in Step S9 is then ended.

After Step S9 described above, the determined result display image data generating unit 52 generates determined result display image data using the B mode image data generated at the B mode image data generating unit 51, the feature data calculated at the feature data extracting unit 43, and the determined result determined at the tissue characteristic determining unit 44 (Step S10).

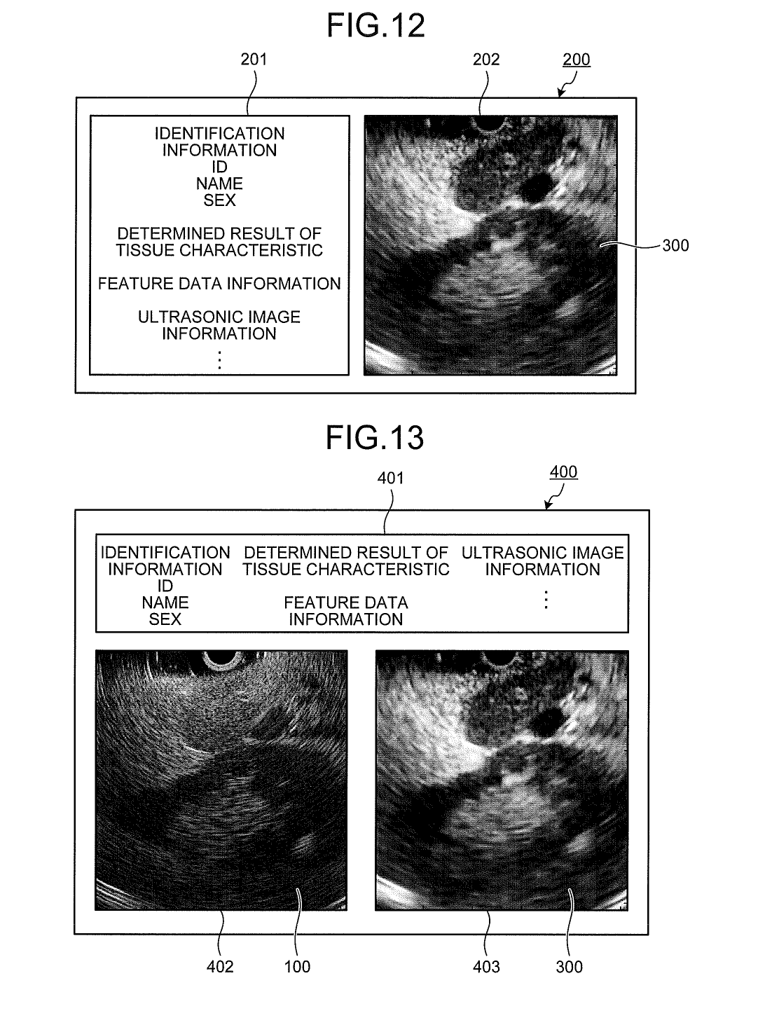

After the data generation, the display unit 7 displays the determined result display image generated at the determined result display image data generating unit 52 (Step S11). FIG. 12 is a diagram of an exemplary display of a determined result display image displayed on the display unit 7. A determined result display image 200 illustrated in FIG. 12 includes an information display unit 201 on which various related items of information including the determined result of a tissue characteristic are displayed and an image display unit 202 on which a tissue characteristic emphasized image that emphasizes a tissue characteristic is displayed based on the B mode image.

On the information display unit 201, the identification information (an ID number, a name, a sex, or the like) of a specimen, the determined result of the tissue characteristic calculated at the tissue characteristic determining unit 44, information about feature data in determining the tissue characteristic, and ultrasonic image quality information such as a gain and contrast are displayed, for example. Here, for information about feature data, display can be performed using the average and standard deviation of the feature data of the frequency spectra of Q sets of FFT data groups located in the area of interest. More specifically, on the information display unit 201, a=1.5±0.3 (dB/MHz), b=−60±2 (dB), and c=−50±1.5 (dB) can be displayed, for example.

A tissue characteristic emphasized image 300 displayed on the image display unit 202 is a gray scale image in which the intercept b is equally allocated to R (red), G (Green), and B (blue) in the B mode image 100 in FIG. 4.

The display unit 7 displays the determined result display image 200 in the configuration above, and the operator can more accurately grasp a tissue characteristic in the area of interest.

It is noted that the tissue characteristic emphasized image 300 illustrated in FIG. 12 is merely an example. In addition to this, the slope a, the intercept b, and the intensity c may be allocated to R (red), G (Green), and B (blue) to also display a tissue characteristic emphasized image in a color image, for example. In this case, since tissue characteristics are expressed in unique colors, the operator can grasp a tissue characteristic in the area of interest based on the color distribution of the image. Moreover, such a configuration may be possible in which the color space is configured of complementary color variables such as cyan, magenta, and yellow for allocating feature data to the variables, instead of configuring the color space in an RGB color system. Furthermore, tissue characteristic emphasized image data may be generated by mixing B mode image data with color image data at a predetermined ratio. In addition, tissue characteristic emphasized image data may be generated by replacing only the area of interest with color image data.

According to the first embodiment of the present invention described above, in approximating the frequency spectrum obtained by analyzing the frequency of received ultrasonic waves, the feature data of the frequency spectrum of the frequency band set to the individual receiving depths of ultrasonic waves is extracted, and this feature data is used as well as feature data of a frequency spectrum extracted based on ultrasonic waves reflected off a plurality of known specimens are used for determining a tissue characteristic in a predetermined area in the specimen, so that difference in tissue can be clearly distinguished without using the strain value and the modulus of elasticity of human tissue. Accordingly, it is possible to accurately discriminate tissue characteristics, and it is possible to improve the reliability of measured results.

Moreover, according to the first embodiment, a frequency band is set in such a way that the bandwidth is narrower and the maximum frequency is smaller as the receiving depth is deeper, so that it is possible to eliminate the influence of attenuation in association with the propagation of ultrasonic waves, and it is possible to more highly accurately determine a tissue characteristic.

FIG. 13 is a diagram of another exemplary display of a determined result display image on the display unit 7. A determined result display image 400 illustrated in FIG. 13 has an information display unit 401, a first image display unit 402 on which a B mode image is displayed, and a second image display unit 403 on which a tissue characteristic emphasized image is displayed. In the case illustrated in FIG. 13, the B mode image 100 is displayed on the first image display unit 402, and the tissue characteristic emphasized image 300 is displayed on the second image display unit 403. The B mode image and the tissue characteristic emphasized image are thus displayed side by side, so that differences between the two images can be recognized on a single screen. Incidentally, an image displayed on the first image display unit 402 may be replaced with an image displayed on the second image display unit 403. Moreover, the display of the determined result display image 200 and the display of the determined result display image 400 may be switched through an input from the input unit 6.

Incidentally, in the first embodiment, the frequency band setting unit 42 sets a frequency band for every receiving depth with reference to the frequency band table Tb. However, a frequency band may be set by accepting the setting of the frequency band input at the input unit 6, for example. In this case, since a user can freely change frequency bands through the input unit 6, the frequency band is adjusted for every specimen to reduce differences in the individual specimens.

### Second Embodiment

In a second embodiment of the present invention, a tissue characteristic determination process in a tissue characteristic determining unit is different from the first embodiment. The configuration of an ultrasonic diagnosis apparatus according to the second embodiment is the same as the configuration of the ultrasonic diagnosis apparatus 1 described in the first embodiment. Therefore, in the following description, components corresponding to the components of the ultrasonic diagnosis apparatus 1 are designated the same reference numerals and signs.

A tissue characteristic determining unit 44 adds the feature data (a, b, and c) of Q sets of FFT data groups located in the area of interest to groups SA, SB, and SC (see FIG. 11) configuring tissue characteristics A, B, and C to form a new population, and then determines the standard deviation for every item of feature data of data configuring tissue characteristics.

After the determination, the tissue characteristic determining unit 44 calculates a difference between the standard deviations of the items of feature data of groups SA, SB, and SC in the original population formed of only known specimens and the standard deviations of the items of feature data of groups SA, SB, and SC in a new population added with a new specimen (in the following, simply referred to as “a standard deviation difference”), and determines a tissue characteristic corresponding to a group including feature data with the smallest standard deviation difference as a tissue characteristic of a specimen.

Here, the tissue characteristic determining unit 44 may calculate a standard deviation difference only on the standard deviation of feature data selected in advance from a plurality of items of feature data. The operator may freely select feature data in this case, or an ultrasonic diagnosis apparatus 1 may automatically select feature data.

Moreover, such a configuration may be possible in which the tissue characteristic determining unit 44 calculates a value that the standard deviation differences of all the items of feature data are appropriately weighted and added for every group and determines a tissue characteristic corresponding to a group that this value is the minimum as a tissue characteristic of a specimen. In this case, when the items of feature data are the slope a, the intercept b, and the intensity c, for example, the tissue characteristic determining unit 44 calculates wₐ (the standard deviation difference of a)+w_{b}•(the standard deviation difference of b)+w_{c}•(the standard deviation difference of c) where weights for the slope a, the intercept b, and the intensity c are wₐ, w_{b}, and w_{c}, respectively, and determines the tissue characteristic of a specimen based on the calculated value. It is noted that the values of the weights wₐ, w_{b}, and w_{c} may be freely set by the operator, or may be automatically set by the ultrasonic diagnosis apparatus 1.

Moreover, such a configuration may be possible in which the tissue characteristic determining unit 44 calculates the square root of a value that the second power of the standard deviation differences of all the items of feature data is appropriately weighted and added for every group and determines a tissue characteristic corresponding to a group that this square root is the minimum as a tissue characteristic of a specimen. In this case, when the items of feature data are the slope a, the intercept b, and the intensity c, for example, the tissue characteristic determining unit 44 calculates {w′ₐ•(the standard deviation difference of a)²+w′_{b}•(the standard deviation difference of b)²+w′_{c}•(the standard deviation difference of c)²}^{1/2} where weights for the slope a, the intercept b, and the intensity c are w′ₐ, w′_{b}, and w′_{c}, respectively, and determines a tissue characteristic based on the calculated value. It is noted that also in this case, the values of the weights w′ₐ, w′_{b}, and w′_{c} may be freely set by the operator, or may be automatically set by the ultrasonic diagnosis apparatus 1.

According to the second embodiment of the present invention described above, it is possible to accurately discriminate tissue characteristics, and it is possible to improve the reliability of measured results as similar to the first embodiment described above.

It is noted that in the second embodiment, the tissue characteristic determining unit 44 determines a tissue characteristic based on a change in the standard deviations of the items of feature data between the original population and the population added with a new specimen, and this is merely an example. For example, the tissue characteristic determining unit 44 may determine a tissue characteristic based on a change in the averages of the items of feature data between the original population and the population added with a new specimen.

### Third Embodiment

In a third embodiment of the present invention, a tissue characteristic determination process in a tissue characteristic determining unit is different from the first embodiment. The configuration of an ultrasonic diagnosis apparatus according to the third embodiment is the same as the configuration of the ultrasonic diagnosis apparatus 1 described in the first embodiment. Therefore, in the following description, components corresponding to the components of the ultrasonic diagnosis apparatus 1 are designated the same reference numerals and signs.

A tissue characteristic determining unit 44 calculates probabilities belonging to tissue characteristics using distances between the average mark of a specimen in a feature data space and the average marks of the tissue characteristics of known specimens. More specifically, in the case of the feature data space (b, c) illustrated in FIG. 11, the distances α, β, and γ between the specimen average mark Sp and the known specimen average marks A₀, B₀, and C₀ are used to calculate probabilities belonging to tissue characteristics. Probabilities belonging to known specimens are set in such a way that a smaller distance has a greater probability. For example, a probability belonging to a tissue characteristic A can be defined as λ/α (%), a probability belonging to a tissue characteristic B can be defined as λ/β (%), and a probability belonging to a tissue characteristic C can be defined as λ/γ (%), where λ=100/(α⁻¹+β⁻¹+γ⁻¹) (%).

In the third embodiment, in displaying a determined result display image on a display unit 7, probabilities belonging to tissue characteristics are displayed on an information display unit. For example, in the case where the display unit 7 displays the determined result display image 200, the information display unit 201 displays the determined result as “a probability that a tissue characteristic is A=60%, a probability that a tissue characteristic is B=5%, and a probability that a tissue characteristic is C=35%”.

According to the third embodiment of the present invention described above, it is possible to accurately discriminate tissue characteristics, and it is possible to improve the reliability of measured results as similar to the first embodiment described above.

### Fourth Embodiment

In a fourth embodiment of the present invention, a tissue characteristic determination process in a tissue characteristic determining unit is different from the first embodiment. The configuration of an ultrasonic diagnosis apparatus according to the forth embodiment is the same as the configuration of the ultrasonic diagnosis apparatus 1 described in the first embodiment. Therefore, in the following description, components corresponding to the components of the ultrasonic diagnosis apparatus 1 are designated the same reference numerals and signs.

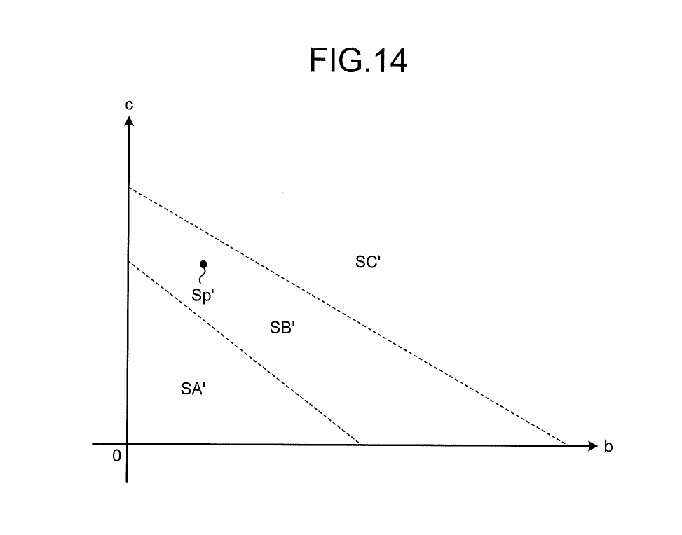

FIG. 14 is a diagram of the outline of a process for determining a tissue characteristic performed at a tissue characteristic determining unit 44 according to the forth embodiment. In a feature data space illustrated in FIG. 14, the horizontal axis expresses the intercept b, and the vertical axis expresses the intensity c. In this feature data space, areas are grouped according to tissue characteristics. The tissue characteristic determining unit 44 determines tissue characteristics according to locations of specimen average marks. FIG. 14 is the case where a specimen average mark Sp′ belongs to a group SB′ (an area that a tissue characteristic is B). In this case, the tissue characteristic determining unit 44 determines that a tissue characteristic in the area of interest of a specimen is B.

According to the fourth embodiment of the present invention described above, it is possible to accurately discriminate tissue characteristics, and it is possible to improve the reliability of measured results as similar to the first embodiment described above.

The embodiments for carrying out the present invention have been described so far. The present invention should not be limited only by the foregoing first to fourth embodiments. Namely, the present invention can include various embodiments in the scope not deviating from the technical idea of the appended claims.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic diagnosis apparatus that transmits an ultrasonic wave to a specimen and receives an ultrasonic wave reflected off the specimen for determining a tissue characteristic of the specimen based on a received ultrasonic wave, the ultrasonic diagnosis apparatus comprising:
a frequency analyzing unit configured to calculate a frequency spectrum by analyzing a frequency of a received ultrasonic wave;
a frequency band setting unit configured to set at least an upper limit frequency of a frequency band for use in approximating the frequency spectrum calculated at the frequency analyzing unit to a predetermined frequency according to a receiving depth of an ultrasonic wave;
a feature data extracting unit configured to extract feature data of the frequency spectrum by approximating a frequency spectrum of the frequency band set at the frequency band setting unit;
a storage unit configured to store feature data of a frequency spectrum extracted based on ultrasonic waves reflected off a plurality of known specimens in association with tissue characteristics of the plurality of known specimens; and
a tissue characteristic determining unit configured to determine a tissue characteristic in a predetermined area in the specimen using feature data stored in the storage unit in association with tissue characteristics of the plurality of known specimens and the feature data extracted at the feature data extracting unit.

2. The ultrasonic diagnosis apparatus according to claim 1, wherein the frequency band setting unit further sets a lower limit frequency of the frequency band according to a receiving depth of an ultrasonic wave.

3. The ultrasonic diagnosis apparatus according to claim 2, wherein:
the frequency band setting unit includes a frequency band storage unit configured to store at least one of an upper limit and a lower limit of a frequency band for use in approximating the frequency spectrum according to the receiving depth; and
the frequency band setting unit reads corresponding information out of the frequency band storage unit according to the receiving depth of an ultrasonic wave, and sets the frequency band.

4. The ultrasonic diagnosis apparatus according to claim 1, wherein the frequency band is determined according to a receiving depth of an ultrasonic wave, and a bandwidth is narrower and an upper limit frequency is smaller as the receiving depth is deeper.

5. The ultrasonic diagnosis apparatus according to claim 1, comprising an input unit configured to accept an input of a setting of a frequency band,
wherein the frequency band setting unit sets a frequency band based on information accepted at the input unit.

6. The ultrasonic diagnosis apparatus according to claim 1, wherein the feature data extracting unit approximates the frequency spectrum in a polynomial equation by regression analysis.

7. The ultrasonic diagnosis apparatus according to claim 6, wherein:
the feature data extracting unit approximates the frequency spectrum in a linear expression; and
the feature data extracting unit extracts a plurality of items of feature data including at least two of a slope of the linear expression, an intercept of the linear expression, and intensity determined using the slope, the intercept, and a specific frequency included in a frequency band of the frequency spectrum.

8. The ultrasonic diagnosis apparatus according to claim 7, wherein:
the storage unit stores an average of items of feature data of groups sorted according to tissue characteristics with respect to the plurality of known specimens; and
the tissue characteristic determining unit sets a feature data space having at least one of the plurality of items of feature data as a component, and determines a tissue characteristic of the specimen based on a distance on the feature data space between a specimen average mark having an average of feature data forming components of the feature data space in feature data of a frequency spectrum in a predetermined area in the specimen as coordinates of the feature data space and a known specimen average mark having an average of feature data forming components of the feature data space in items of feature data of groups of the plurality of known specimens as coordinates of the feature data space.

9. The ultrasonic diagnosis apparatus according to claim 1, wherein the tissue characteristic determining unit calculates a standard deviation of feature data in a population that feature data of the specimen is added to groups sorted according to tissue characteristic of the plurality of known specimens, and determines a tissue characteristic corresponding to a group having feature data that a difference between the standard deviation and a standard deviation of feature data in the groups as a tissue characteristic of the specimen.

10. The ultrasonic diagnosis apparatus according to claim 1, further comprising a determined result display image data generating unit configured to generate visual information corresponding to feature data of the specimen and to generate determined result display image data to display a determined result of a tissue characteristic in a predetermined area in the specimen using the generated visual information, an image generated based on a received ultrasonic wave, and a result determined at the tissue characteristic determining unit.

11. The ultrasonic diagnosis apparatus according to claim 10, wherein the visual information is a variable configuring a color space.

12. An operation method of an ultrasonic diagnosis apparatus that transmits an ultrasonic wave to a specimen and receives an ultrasonic wave reflected off the specimen for determining a tissue characteristic of the specimen based on a received ultrasonic wave, the operation method comprising:
calculating a frequency spectrum at a frequency analyzing unit by analyzing a frequency of a received ultrasonic wave;
setting at least an upper limit frequency of a frequency band for use in approximating the calculated frequency spectrum to a predetermined frequency according to a receiving depth of an ultrasonic wave;
extracting feature data of the frequency spectrum at a feature data extracting unit by approximating a frequency spectrum of the set frequency band; and
determining a tissue characteristic in a predetermined area in the specimen at a tissue characteristic determining unit using feature data read out of a storage unit storing feature data of a frequency spectrum extracted based on ultrasonic waves reflected off a plurality of known specimens in association with tissue characteristics of the plurality of known specimens and the feature data extracted at the feature data extracting unit.

13. A non-transitory computer readable recording medium with an executable program recorded thereon, wherein the program instructs a processor to perform:
calculating a frequency spectrum at a frequency analyzing unit by analyzing a frequency of a received ultrasonic wave;
setting at least an upper limit frequency of a frequency band for use in approximating the calculated frequency spectrum to a predetermined frequency according to a receiving depth of an ultrasonic wave;
extracting feature data of the frequency spectrum at a feature data extracting unit by approximating a frequency spectrum of the set frequency band; and
determining a tissue characteristic in a predetermined area in the specimen at a tissue characteristic determining unit using feature data read out of a storage unit storing feature data of a frequency spectrum extracted based on ultrasonic waves reflected off a plurality of known specimens in association with tissue characteristics of the plurality of known specimens and the feature data extracted at the feature data extracting unit.
